(19) United States
(12) Patent Application Publication
(10) Pub. No.: US 2005/0021375 A1
(43) Pub. Date: Jan. 27, 2005

(54) Cooperative diagnosis system

(76) Inventors: Satoshi Shimizu, Kanagawa (JP); Kyoji Sekiguchi, Tochigi (JP); Hiroki Uchida, Tochigi (JP)

Correspondence Address: MORGAN & FINNEGAN, L.L.P., 3 WORLD FINANCIAL CENTER, NEW YORK, NY, 10281-2101, US

(21) Appl. No.: 10/919,778

(22) Filed: Aug. 16, 2004

Related U.S. Application Data
(63) Continuation of application No. PCT/JP03/04230, filed on Apr. 2, 2003.

(30) Foreign Application Priority Data
Aug. 4, 2003 (JP) 2002-102308
Aug. 4, 2003 (JP) 2002-102309
Aug. 9, 2003 (JP) 2002-106906

(51) Int. Cl. G06F 17/60; A61B 5/00

(52) U.S. Cl. 705/2; 600/300

(57) ABSTRACT

A cooperative diagnosis system for allowing a plurality of doctors to efficiently make a diagnosis in cooperation with each other is provided. When a terminal apparatus 112 on a requesting side 110 issues a diagnosis request to a server 120, the server 120 selects a doctor in charge of a diagnosis in accordance with preset priority levels, and transmits diagnosis request mail to the selected doctor's address in charge of the diagnosis. The doctor in charge of the diagnosis accesses the server 120 from his/her terminal 131A to browse examination data and register a diagnosis result in the server 120. The doctor on the requesting side 110 accesses the server 120 by using his/her terminal 112 to browse the diagnosis result.

### TECHNICAL FIELD

The present invention relates to a cooperative diagnosis system in which a plurality of doctors cooperatively make a diagnosis.

### BACKGROUND ART

Conventionally, in making a diagnosis, one doctor generally examines one patient. In addition, since one doctor has a limit to his/her ability or experience, he/she often requires a second opinion from another doctor or refers the patient to another hospital and entrusts a diagnosis to another doctor.

When, for example, a diabetic patient visits a physician, it may be diagnosed that the patient may have diabetic retinopathy. The physician cannot accurately determine diabetic retinopathy, and thence the patient preferably takes a medical examination from an ophthalmologist. However, no ophthalmology department may exit within the same hospital facilities. In such a case, the physician refers the patient to the ophthalmology department of another hospital facilities.

In referring the patient to another ophthalmologist, the above physician must write a letter of introduction and generate an examination data sheet and the like. The physician needs also to communicate with the ophthalmologist to whom the patient is referred. This requires much time and effort. In addition, since the physician and ophthalmologist serve in different hospital facilities, it is cumbersome for the patient to regularly visit the different hospitals.

In order to solve this problem, some physicians use a non-mydriatic fundus camera or the like to take fundus photographs of patient's eyes, and mail a photographed film or the like, thereby entrusting an ophthalmologist in another facilities with a diagnosis based on image interpretation.

In the method of mailing such a film, however, it takes time and trouble to mail data. In addition, images such as films, patient data, examination data, and the report made by the ophthalmologist must be managed together. It therefore requires much labor in terms of clerical work.

Assume that a plurality of doctors are to cooperatively make a diagnosis. In many cases, however, such a diagnosis cannot be made because, for example, the date when the doctor on the requesting side entrusts a diagnosis does not match the requested doctor's schedule, or the case falls outside the specialty of the requested doctor. In such a case, the requested doctor must re-entrust still another doctor with a diagnosis.

Conventionally, however, when the requested doctor is to re-entrust still another doctor with a diagnosis, the requested doctor has full discretion on the selection of a doctor who he/she entrusts with a diagnosis. It is therefore difficult for the hospital on the requesting side to manage the information. For example, a diagnosis may be entrusted to a doctor who is completely not associated with the hospital on the requesting side. This makes it impossible to ensure the security of various data contained in a diagnosis request.

### DISCLOSURE OF INVENTION

Accordingly, it is an object of the present invention to provide a technique which helps a requesting doctor and a requested doctor efficiently make a cooperative diagnosis according to situation in a requested doctor side.

In one aspect, the present invention provides a remote diagnosis system which helps a doctor on a requesting side request a remote diagnosis to a doctor on a requested side to execute the remote diagnosis. The system may comprise a component for storing examination data of a patient transmitted from a terminal on a requesting side, a component for selecting a doctor on a requested side on the basis of the examination data of the patient and preset priority levels, a component for transmitting a diagnosis request to the selected doctor on the requested side,a component for transmitting the registered examination data when being accessed by the selected doctor on the requested side from a terminal on the requested side, a component for storing diagnosis result data transmitted from the terminal on the requested side and a component for transmitting the diagnosis result data to the terminal on the requesting side.

In another aspect, the present invention provide a remote system may comprise component for storing diagnosis/treatment information including a medical image transmitted from a terminal on a requesting side, a component for delivering the medical image to a terminal on a requested side; component for acquiring information concerning a reference state of the terminal on the requested side with respect to the delivered medical image and a component for storing the acquired information concerning the reference state.

In furthermore other aspect, the present invention provides a remote diagnosis system which may comprise a component for storing a diagnosis request content transmitted from a terminal on a requesting side and a component for evaluating a display apparatus used by the doctor on the requested side on the basis of the diagnosis request content.

Other features and advantages of the present invention will be apparent from the following description taken in conjunction with the accompanying drawings, in which like reference characters designate the same or similar parts throughout the figures thereof.

### BRIEF DESCRIPTION OF DRAWINGS

The accompanying drawings, which are incorporated in and constitute a part of the specification, illustrate embodiments of the invention and, together with the description, serve to explain the principles of the invention.

FIG. 1 is a view showing a system configuration according to an embodiment;

FIG. 2 is a flow chart showing the flow of processing for a diagnosis request according to the embodiment;

FIG. 3 is a view showing jobs for a diagnosis request according to the embodiment;

FIG. 4 is a view showing an example of a diagnosis request form window according to the embodiment;

FIG. 5 is a view showing an example of a diagnosing doctor selection setting window according to the embodiment;

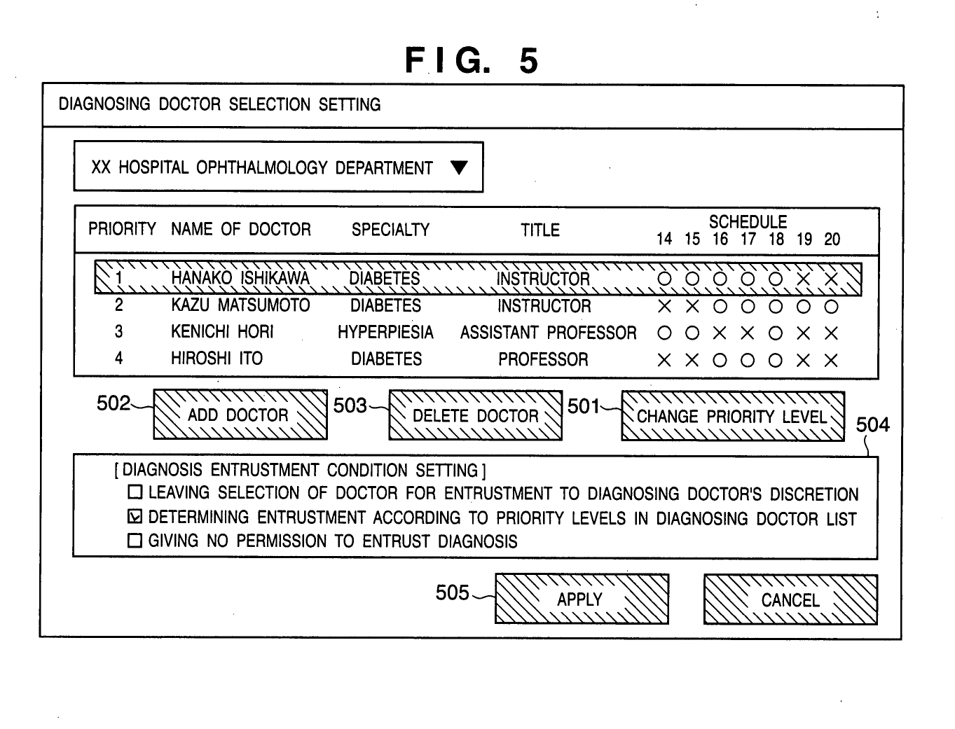

FIG. 6 is a view showing an example of a diagnosis request list window according to the embodiment;

FIG. 7 is a view showing an example of a diagnosis report window according to the embodiment;

FIG. 8 is a view showing an example of a diagnosis report confirmation window according to the embodiment;

FIG. 9 is a flow chart showing the flow of processing for a diagnosis request according to the embodiment;

FIG. 10 is a view showing a remote image diagnosis system according to the embodiment;

FIG. 11 is a view showing an information transmission/reception sequence between a requesting side and a data center according to the embodiment;

FIG. 12 is a view showing an information transmission/reception sequence between a diagnosing side and a data center according to the embodiment;

FIGS. 13A to 13E are views showing images for testing the performance of a display apparatus installed on an diagnosing side according to the embodiment; and

FIG. 14 is a flow chart showing a display apparatus performance test according to the embodiment.

### BEST MODE FOR CARRYING OUT THE INVENTION

Preferred embodiments of the present invention will now be described in detail in accordance with the accompanying drawings.

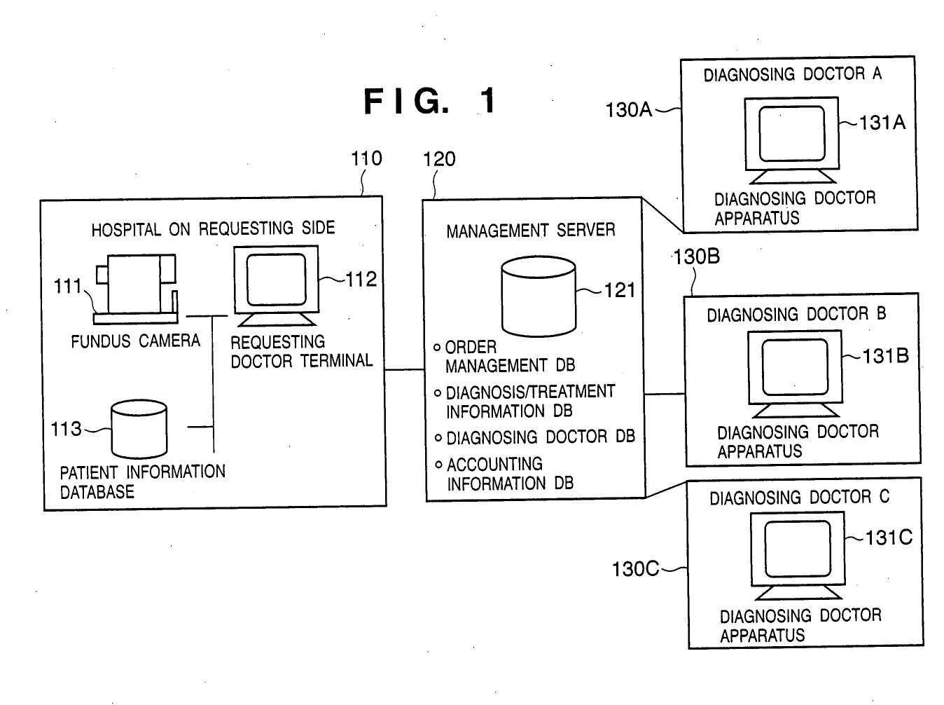

FIG. 1 shows an example of the arrangement of a cooperative diagnosis system according to an embodiment of the present invention. Reference numeral 110 denotes a hospital on a requesting side, which includes a photographing apparatus 111 such as a fundus camera for photographing fundus images and a requesting doctor terminal 112 which records examination information, patient information, and the like together with photographed images to make a request to another doctor (diagnosing doctor). Patient information registered at the time of reception of patients is stored in a patient information database 113. Since the patient information database 113 is connected to the photographing apparatus 111 and requesting doctor terminal 112 through a network, the image data photographed by the photographing apparatus 111 and the like are also stored in the patient information database 113 in association with the patient information. In requesting a diagnosis, therefore, the requesting doctor terminal 112 can generate a diagnosis request form by retrieving patient information and the like from the patient information database 113. The requesting doctor terminal 112 is connected to a management server 120 through a network such as a LAN or the Internet to transmit a diagnosis request form to the management server 120. The management server 120 registers diagnosis request data on the basis of the received diagnosis request form. With this operation, a diagnosis is requested. Note that each terminal and server described in this embodiment are computers each including a CPU, memory, hard disk drive, communication interface, input unit, and the like. Each computer executes the following processing in accordance with a corresponding program.

The management server 120 includes a diagnosis/treatment database, an order management database for managing diagnosis request orders, a diagnosing doctor database for managing registered doctor data, an accounting database for managing accounting/money distribution, and the like. The registered hospital 110 on the requesting side, diagnosing doctors 130A, 130B, and 130C, and the like can connect to the management server 120 through a network and can retrieve and register information from and in the server, as needed. The management server 120 may be installed within the hospital on the requesting side facilities or installed in a place other than the hospital on the requesting side. For example, the management server 120 may be installed on a public network such as the Internet as long as security is sufficiently ensured. In an extreme case, such a server may be installed in a foreign country.

Each of the diagnosing doctors 130A, 130B, and 130C has a diagnosing doctor terminal 131A or the like and can browse a diagnosis request addressed to itself, input a diagnosis result, and register diagnosis result data by transmitting it to the management server 120 by connecting to the management server 120 through a network. A plurality of diagnosing doctors can share one diagnostic terminal 131 by making authentication using digital signatures, passwords, or the like. In this case, an authentication server may be used to manage doctor accounts. An application used for a diagnosis can also be generated on a Web base technology. A Web-based application allows a user to execute necessary processing such as request processing, browse processing, and diagnosis report processing by using a Web browser. As described above, a Web-based application allows a user to process a requested diagnosis from any kind of PC regardless of the type of terminal and OS, providing convenience.

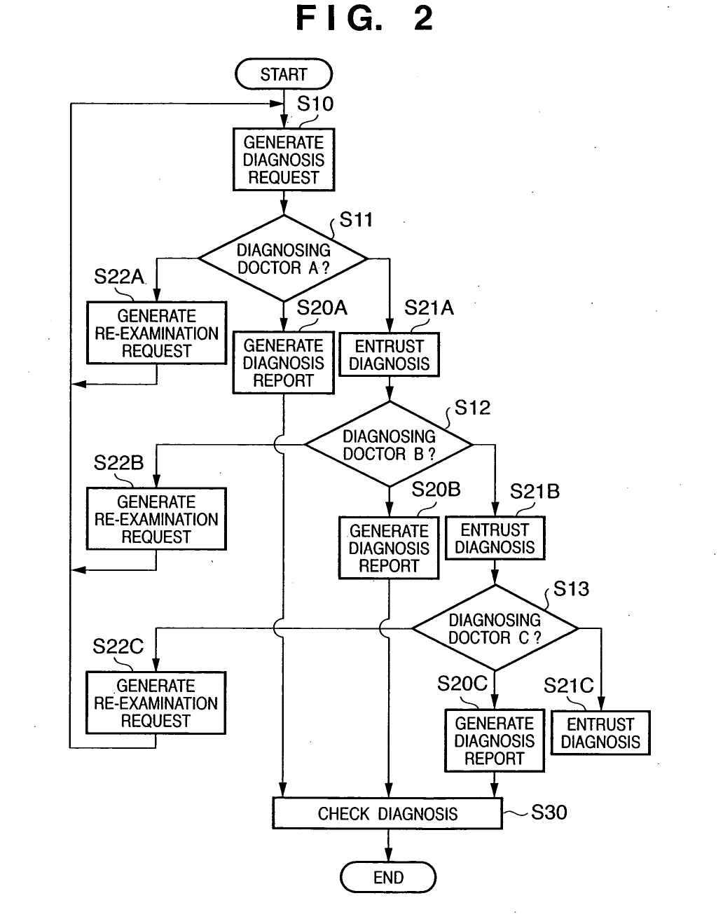

FIG. 2 is a view showing a work flow in a case wherein a plurality of doctors cooperatively make a diagnosis by using this cooperative diagnosis system. In step S10, a hospital on a requesting side issues a diagnosis request to a diagnosing doctor A with the highest priority level through the management server 120. In step S20A, the diagnosing doctor A makes a diagnosis by image interpretation and reports the diagnosis to the hospital on the requesting side. In step S30, the doctor on the requesting side in the hospital on the requesting side checks the diagnosis. With this operation, this examination is finished.

If it is determined in step S21A that the diagnosing doctor A cannot make a diagnosis because the patient case associated with the diagnosis request falls outside the realm of his/her specialty, the diagnosing doctor A entrusts a diagnosis. That is, the diagnosing doctor A can re-entrust another doctor with the diagnosis request issued to the diagnosing doctor A.

If a diagnosis re-entrust request is issued, the management server 120 transmits, in step S21B, a diagnosis entrust request to a diagnosing doctor B with the highest priority level next to the diagnosing doctor A.

Assume that upon reception of diagnosis requests, the diagnosing doctors A and B and a diagnosing doctor C determine in steps S22A, 22B, and 22C, respectively, that they cannot make diagnoses because of the poor quality of diagnosis images, insufficient patient data, and the like. In this case, each doctor can issue a re-examination request to a hospital 10 on a requesting side. A re-examination request may be issued through the management server 120 or directly to the hospital 10 on the requesting side. When a re-examination request is issued, the diagnosis request is canceled. The requesting side is then notified of the corresponding information. With regard to the case for which the re-examination request has been issued, for example, images are photographed again and patient data is added and corrected in the hospital 10 on the requesting side, and a diagnosis request is issued again as a new examination request.

The diagnosis request generated in the hospital 10 on the requesting side in this manner is finished when any diagnosing doctor makes a diagnosis report or the request is canceled as a re-examination request.

Assigning priority levels to diagnosing doctors in this manner allows the hospital on the requesting side to manage routes for a diagnosis request and diagnosis entrustment and to reliably ensure the security of information associated with diagnoses.

If no diagnosis report is made within a set deadline of a diagnosis result report, e.g., within a one week after a diagnosis request, the management server 120 may automatically transmit mail asking for a report to the requested side, transmit a re-examination request to the requesting side, or cancel the diagnosis request. This makes it possible to prevent the diagnosing doctor from stagnating in an examination.

Assume that the upper limit of the number of times of diagnosis entrustment is stored in the management server 120. In this case, when the number of times of diagnosis entrustment is counted and becomes three, a re-examination request may be transmitted to cancel the diagnosis request. With this operation, a difficult case for which diagnosing entrustment is repeated can be automatically canceled even within a time limit. This makes it possible to proceed with the next processing such as another examination without wasting time.

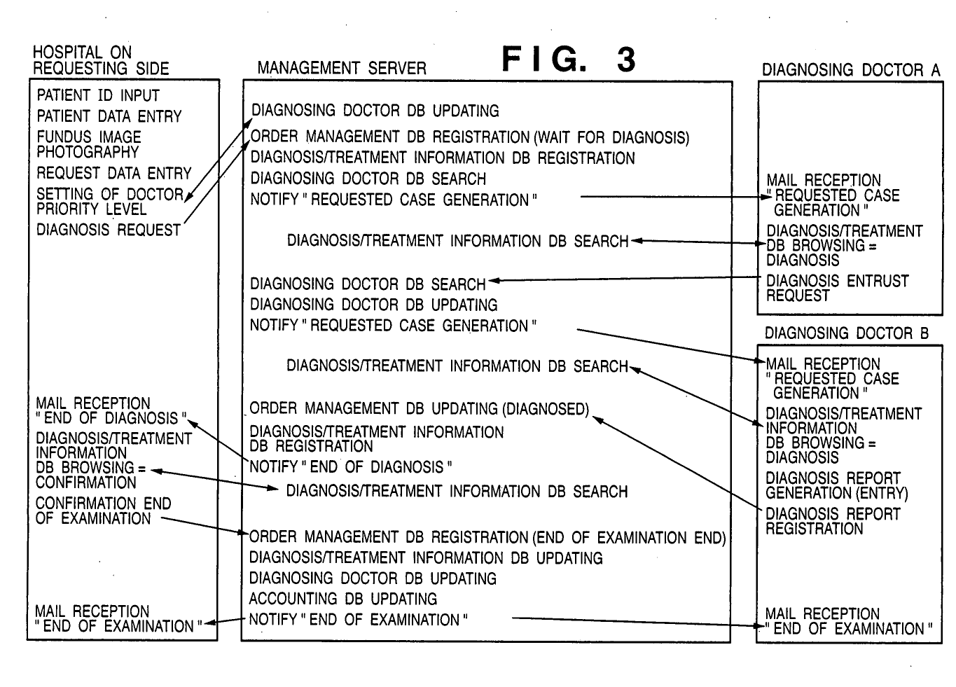

A method of realizing the above work flow will be described in detail. FIG. 3 shows the steps of processing to be performed by a hospital on a requesting side, a diagnosing doctor, and the management server 120.

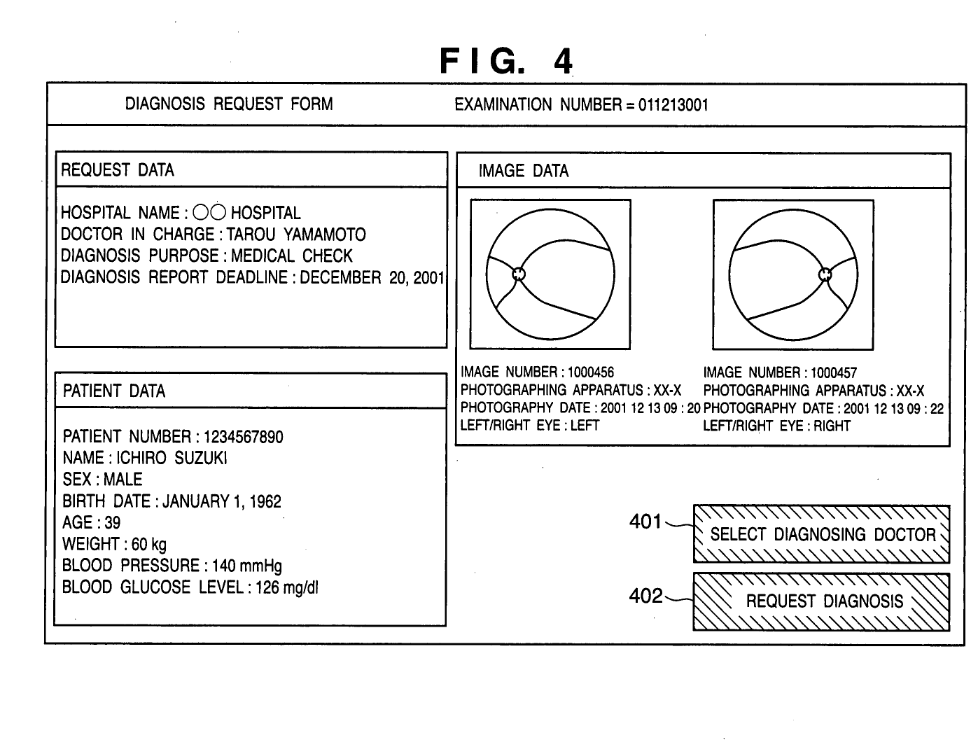

First of all, in the hospital 10 on the requesting side, an operator reads a patient's magnetic card with a magnetic card reader (not shown) connected to the fundus camera 111, and inputs a patient ID. The operator then inputs unique data of the patient, e.g., the name, sex, date of birth, weight, and blood pressure, with a data input unit such as a keyboard mounted on the fundus camera 111. The operator may retrieve these data from the patient information database 113 connected to the fundus camera 111 through a network by using the patient ID as a key, and input the retrieved data. Proper photographing conditions are then set for the fundus camera 111 to photograph fundus images of the patient. The data of the fundus images photographed by the fundus camera 111 are registered in the patient information database 113, together with the image numbers, photographing apparatus, photography date, and information indicating discrimination between the left and right eyes. When the requesting doctor terminal 112 issues an instruction to generate a diagnosis request form, these data are read out from the patient information database 113, and data are properly input in the respective fields of the diagnosis request form, thereby generating a diagnosis request form window like the one shown in FIG. 4. This diagnosis request form window is displayed on the display of the requesting doctor terminal 112.

The doctor on the requesting side inputs request data, e.g., an examination date, the name of a doctor in charge, an examination purpose, and the dead line of a diagnosis report, by using the input unit of the requesting doctor terminal 112. By operating the requesting doctor terminal 112, patient data is added/corrected, as needed. The doctor on the requesting side may input patient data by using the input unit of the requesting doctor terminal 112 through this diagnosis request form window instead of the fundus camera 111.

When a diagnosing doctor selection button 401 is clicked with an input unit such as a mouse, the requesting doctor terminal 112 displays a window for setting a doctor in charge of diagnosis like the one shown in FIG. 15. This setting window includes a diagnosing doctor list and diagnosis entrustment condition setting field.

The diagnosing doctor list includes, for example, the priority levels, names, specialties, titles, and work schedules of diagnosing doctors. These pieces of information constituting the diagnosing doctor list are also registered in a diagnosing doctor database 121 of the management server 120.

The hospital on the requesting side can set the priority levels of diagnosing doctors in advance in the diagnosing doctor database 121 of the management server 120. The priority levels of diagnosing doctors can be changed by operating a priority level changing button 501 in the diagnosing doctor selection setting window. For example, the priority levels can be changed in accordance with a patient case or in consideration of the schedules of displayed diagnosing doctors. A new diagnosis doctor is added to the diagnosing doctor list by operating a diagnosing doctor add button 502. By operating a delete button 503, a designated diagnosing doctor of the diagnosing doctors registered in the diagnosing doctor database 121 is deleted.

As described above, a diagnosis request from the hospital on the requesting side is issued to a diagnosing doctor with the highest priority level. Conditions under which the diagnosing doctor can further entrust another doctor with a diagnosis can be set in a diagnosis entrustment condition setting field 504.

For example, the requesting side sets one of the following choices:
(1) leaving the selection of a doctor for re-entrustment to the diagnosing doctor's discretion;
(2) determining re-entrustment according to the priority levels in the diagnosing doctor list; and
(3) giving no permission to entrust a diagnosis.

When (1) is selected by the requesting side, the diagnosing doctor can freely select a doctor when further entrusting another doctor with a diagnosis. When (2) is selected, the diagnosing doctor re-entrusts a doctor with a diagnosis in the decreasing order of priority levels in the diagnosing doctor list. When re-entrustment is performed in this manner, since the doctor list designated by the hospital on the requesting side is used, a diagnosis request or diagnosis re-entrust request is issued to only a pre-designated doctor. This makes it possible to ensure the security of diagnosis/treatment data and the like more reliably. When (3) is selected, the diagnosing doctor can re-entrust no doctor with a diagnosis, and hence this diagnosis request is canceled, and the hospital on the requesting side is notified of the corresponding information. If, for example, settings are made to issue a diagnosis request to only one diagnosis doctor but issue no diagnosis request to other diagnosing doctors, more reliable security protection can be realized. On the other hand, if the designated doctor, i.e., the doctor with the highest priority level, cannot make a diagnosis due to inconvenience, the diagnosis request is canceled. In this case, the hospital on the requesting side must designate another doctor and issue another diagnosis request. If, therefore, importance is to be attached to efficiency, a plurality of diagnosing doctors are preferably registered in advance.

When an application button 505 is clicked after the settings in the diagnosing doctor setting window are changed, the diagnosis request form window is restored, and at the same time, the information updated in the diagnosing doctor database 121 of the management server 120 is registered. Subsequent diagnosis requests are processed in accordance with the updated information. If no change is made in the setting associated with diagnosing doctors, this step can be omitted.

After changing the settings associated with the diagnosing doctors, the doctor on the requesting side returns to the diagnosis request form window to click a diagnosis request button 402 upon checking input request data and the like. As a consequence, the contents of the diagnosis request form are transmitted to the management server 120, and the diagnosis request is registered in the management server 120. With this operation, the diagnosis request job is finished.

The management server 120 checks defects in the contents of the respective data items of the transmitted diagnosis request form data. If, for example, a necessary input item is blank, it is determined that there is a defect. If there is no defect, the diagnosis request form data is registered in the diagnosis/treatment information database. At the same time, the examination number of this examination is registered in the order management database, and data such as the occurrence date of the diagnosis request and the status of the current examination (wait for diagnosis) are recorded.

The management server 120 extracts a diagnosing doctor with the highest priority level set in the diagnosing doctor selection list in association with this examination from the diagnosing doctor database. The management server 120 makes contact with the extracted diagnosing doctor (e.g., the diagnosing doctor A) by generating and transmitting e-mail indicating that a diagnosis request has been issued. On the other hand, the management server 120 changes the setting of access level to the diagnosis/treatment database to “browsing/reporting permitted”.

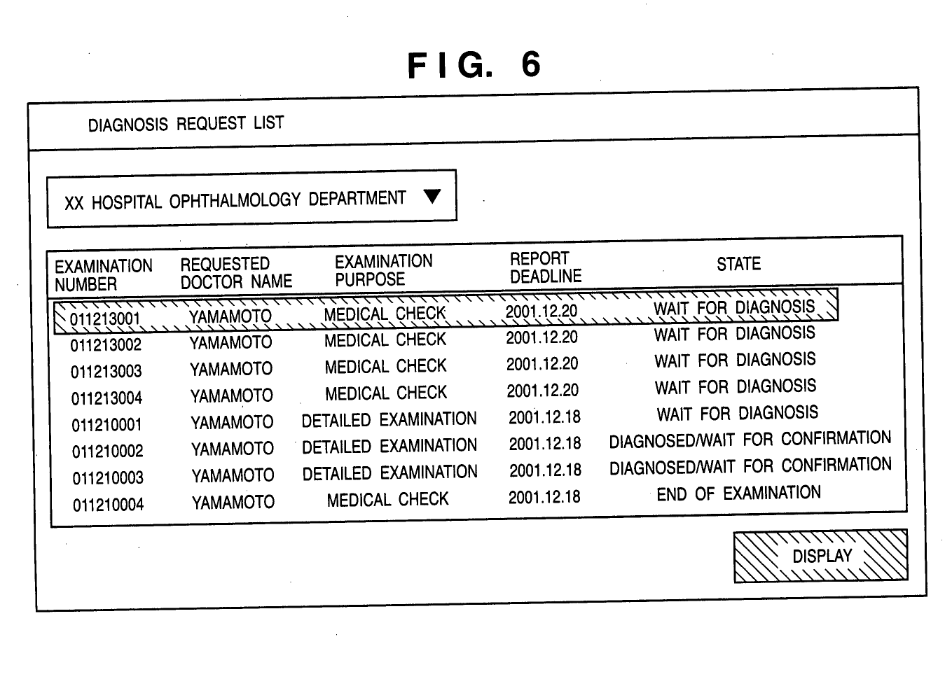

When the diagnosing doctor A knows the generation of the diagnosis request addressed to himself/herself by receiving the e-mail for the diagnosis request, he/she accesses the management server 120 through the diagnosing doctor terminal 131A. The management server 120 authenticates the diagnosing doctor A by using the ID, password, electronic signature, and the like. After the diagnosing doctor A is authenticated, the diagnosis request list shown in FIG. 6 is displayed on the diagnosing doctor terminal 131A. Note that authentication with a fingerprint, iris, or the like can reduce fraud such as a case wherein someone browses diagnosis/treatment data or makes a diagnosis report by disguising himself/herself as the diagnosing doctor A.

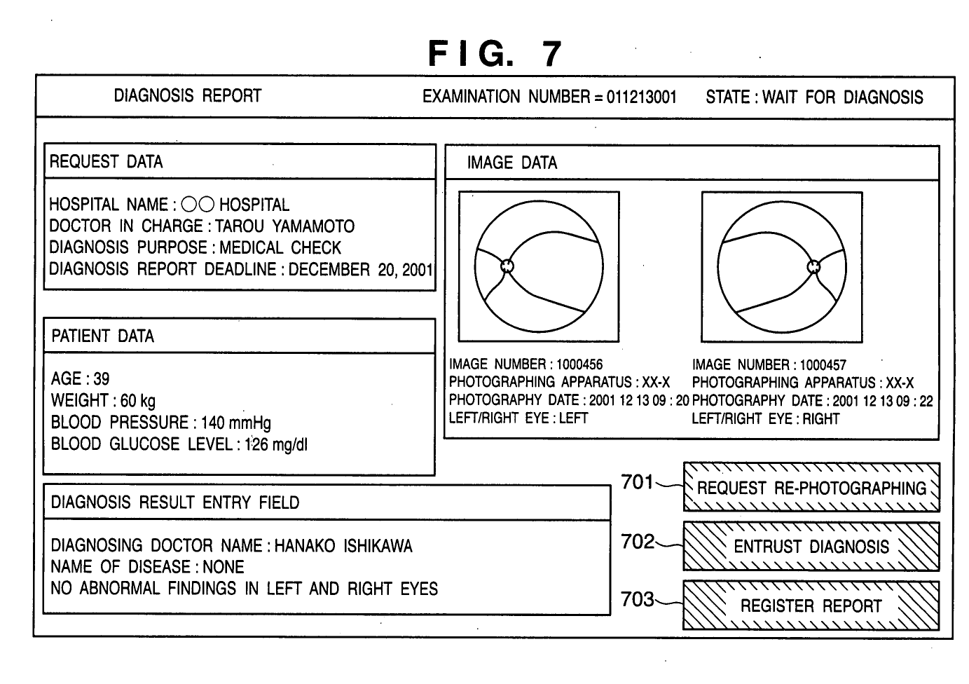

When the diagnosing doctor A selects and displays one of the examination requests from the diagnosis request list displayed on the terminal 131A, a diagnosis report formation window like the one shown in FIG. 7 is displayed on the display of the terminal 131A. In this window, data necessary for a diagnosis are displayed, and an entry field in which the diagnosing doctor is to input a diagnosis result is also displayed. In this entry field, the result obtained by interpreting/diagnosing the examination data of the patient is input.

The data which are displayed to the diagnosing doctor for a diagnosis include, for example, examination data, patient data, image data, and the like. The order management database of the management server 120 has made settings for display/non-display of each item of these data. For example, in order to ensure patient's privacy, the hospital on the requesting side can manage patient data so as to allow the diagnosing doctor to browse only data necessary for a diagnosis, e.g., the age, blood pressure, and blood glucose level without disclosing the name, date of birth, and the like of the patient data.

By operating one of the following buttons included in this window: a re-photographing request button 701, diagnosis re-entrust button 702, and report register button 703, the diagnosing doctor A can execute processing corresponding to the operated button.

When the diagnosing doctor A inputs the diagnosis result in the result entry field and clicks the report register button 703, the terminal 131A transmits the data of the diagnosis report to the management server 120. With this operation, the step for the diagnosis report is completed.

Upon determining that the quality of a displayed image is not sufficient for an image interpretation/diagnosis or there is some suspicion in patient data such as the blood pressure and blood glucose level, the diagnosing doctor A clicks the re-photographing request button 701 to cancel this diagnosis request. This cancellation is registered, together with its reason, in the management server 120 through the terminal 131A. Upon reception of a re-photography request, the management server 120 transfers the re-photography request to the hospital 10 on the requesting side. This re-photography request contains the reason for the cancellation input by the diagnosing doctor.

When the diagnosing doctor A determines that he/she cannot diagnose the requested case because it falls outside the realm of his/her specialty or should entrust another doctor with a diagnosis because of a lack of confidence in a diagnosis, he/she clocks the diagnosis re-entrust button 702. The terminal 131A transmits information indicating the selection of re-entrustment to the management server 120. The management server 120 retrieves/extracts a diagnosing doctor with the next highest priority level from the diagnosis doctor database, and re-entrusts the extracted diagnosing doctor with a diagnosis. This allows the diagnosing doctor A to re-entrust another diagnosing doctor with the diagnosis request addressed to himself/herself.

Note that if the doctor on the requesting side has set the diagnosing entrustment condition in the diagnosing doctor selection setting window to “(3) giving no permission to entrust a diagnosis”, the operation of this button 702 is inhibited. Assume that “(1) leaving the selection of a doctor for entrustment to the diagnosing doctor's discretion” has been selected. In this case, when the button 702 is pressed, the diagnosing doctor list for re-entrustment is displayed on the terminal 131A to allow the diagnosing doctor A to select another doctor in consideration of the case, schedules, and the like.

The flow of processing in which the diagnosing doctor A makes re-entrustment for a diagnosis will be described as an example. A case wherein “(2) determining entrustment according to the priority levels in the diagnosing doctor list” has been selected in diagnosis entrustment setting will be described below.

When the diagnosing doctor A clicks the diagnosis re-entrust button 702, the corresponding information is transmitted to the management server 120. The management server 120 changes the access level setting for the diagnosis/treatment database of the diagnosing doctor A for this examination to “access inhibited”. The management server 120 then extracts a diagnosing doctor with the second highest priority level from the diagnosing doctor database, and changes the access level setting to “browsing/reporting permitted”. Assume that the diagnosing doctor B is ranked second. The management server 120 informs the diagnosing doctor B of the generation of a diagnosis request by using a transmission means such as e-mail.

Upon grasping the generation of the diagnosis request, the diagnosing doctor B entrusted to a diagnosis accesses the management server 120 through a diagnosing doctor terminal 131B. Upon authenticating the diagnosing doctor B from the ID, password, or the like, the management server 120 displays a diagnosis request list like the one shown in FIG. 6, and transmits information about a diagnosis report window like the one shown in FIG. 7 with respect to a selected examination to the terminal 131B as in the case of the diagnosing doctor A.

Like the diagnosing doctor A, the diagnosing doctor B can perform one of a re-photography request, diagnosis re-entrustment, and report registration. If the diagnosing doctor B clicks diagnosis entrustment, a diagnosis request is issued to a diagnosing doctor (diagnosing doctor C) with the next highest priority level, and similar processing is repeated.

A procedure by which a diagnosing doctor registers a report will be described next. The diagnosing doctor browses image data, patient data, and the like in a diagnosis report window like the one shown in FIG. 7, and makes a diagnosis based on these data. The diagnosing doctor then inputs the diagnosis result including the name of a disease, findings, and the like in the diagnosis result entry field. When the report register button 703 is clicked, the input diagnosis result data is transmitted from the terminal 131A to the management server 120.

Upon determining the absence of defects such as blank fields in the transmitted data, the management server 120 registers the diagnosis result in the diagnosis/treatment database, and changes the status on the order management database to “end of diagnosis/wait for confirmation”. The management server 120 informs the hospital on the requesting side of the generation of a diagnosis report through e-mail or the like.

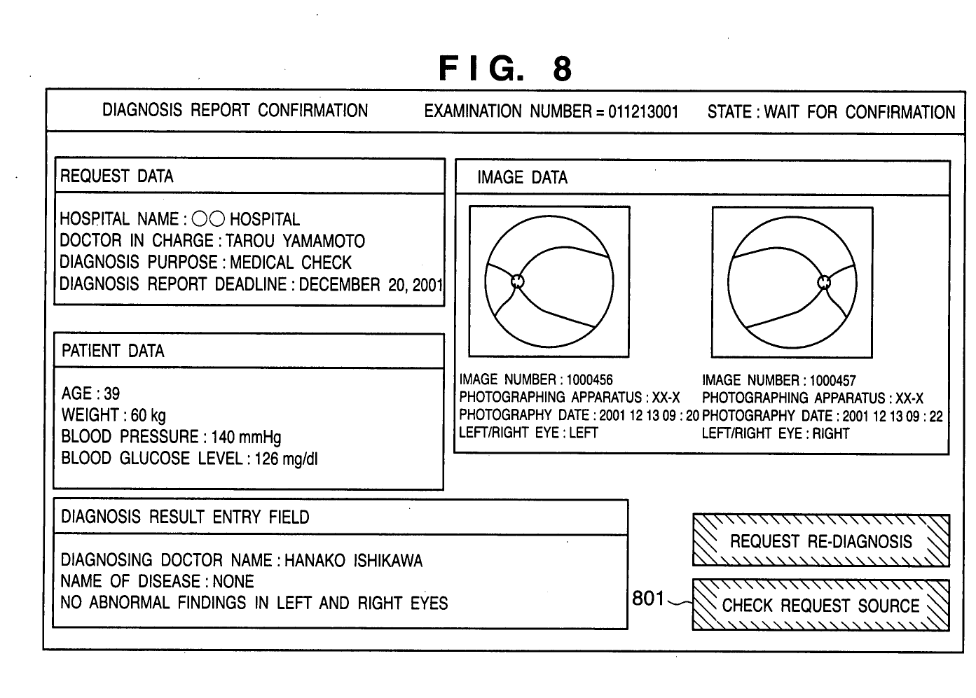

Upon reception of the diagnosis report mail, the doctor on the requesting side accesses the management server 120 by using the requesting doctor terminal 112 in the hospital 10 on the requesting side. As a consequence, a diagnosis report confirmation like the one shown in FIG. 8 is displayed on the display of the terminal 112. When the doctor on the requesting side confirms the contents of the diagnosis result and clicks the comparison start button 801, the corresponding information is transmitted to the management server 120.

The management server 120 updates the examination status of the order management database to “end of examination”, registers the diagnosis report record in the diagnosing doctor database, and registers accounting/money distribution data in the accounting database. Finally, the management server 120 transmits e-mail indicating the completion of all operations for this examination to the hospital 10 on the requesting side and the diagnosing doctor who has made the diagnosis. With the above procedure, the remote diagnosis is terminated.

In the above embodiment, the priority levels of diagnosing doctors are set in the hospital on the requesting side. If, however, these priority levels are automatically updated on the basis of schedules, past records, and the like, diagnosis request processing can be done more efficiently.

For example, data such as the number of diagnosis requests received by each diagnosing doctor, the number of diagnosis reports made, the number of times of diagnosis entrustment, the periods of time required for the respective processes are totalized, and the resultant data are reflected in the priority levels in the diagnosing doctor database. For example, a high priority level is preferably assigned to a doctor who exhibits a short processing time and a low frequency of diagnosis entrustment because he/she is suitable for an efficient cooperation diagnosis. Alternatively, if loads of diagnosis concentrate on some particular doctors, their priority levels may be decreased to uniformly assign diagnosis requests to the respective diagnosing doctors. The efficiency of processing can be further increased by changing the priority levels set by the hospital on the requesting side on the basis of these data.

The use of the cooperative diagnosis system described above allows a plurality of doctors to efficiently and reliably make a diagnosis in cooperation with each other. The hospital on the requesting side, in particular, can easily plan an optimal cooperative diagnosis because it can designate the priority levels of doctors to whom diagnoses are requested. In addition, since the hospital on the requesting side give diagnosis requests to only doctors designated by the hospital in a priority level list, the security of data for the diagnoses can be reliably secured.

In the above embodiment, a physician generates a diagnosis request form containing medical image data of a patient and transmits it to an ophthalmologist in another facilities through the management server 120 to allow the ophthalmologist to perform remote image interpretation/diagnosis of retinopathy, thereby reducing the burden of hospital visit on the patient.

In addition, if it can be determined that medical images delivered to a diagnosing doctor are properly diagnosed by the diagnosing doctor, the doctor on the requesting side and patient can be assured.

In the embodiment to be described below, therefore, there is provided a remote diagnosis system which allows a doctor to determine, in a remote diagnosis, whether a medical image is properly referred to.

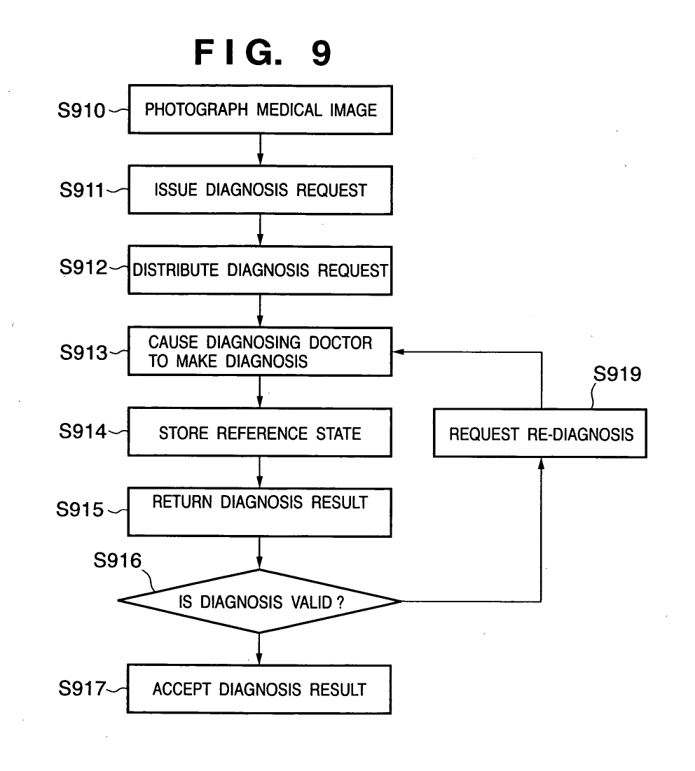

FIG. 9 is a flow chart for a remote diagnosis of a medical image which uses a remote diagnosis system according to this embodiment.

In step S910, in a hospital on a requesting side, an image of an object to be examined is photographed by using a medical photographing apparatus (e.g., a fundus camera 111). The medical image data obtained by photography is stored in a patient information database 113. In addition, e-mail indicating that the medical image data of the patient is stored in the patient information database 113 is transmitted to a requesting doctor terminal 112.

In step S911, the doctor on the requesting side receives the e-mail by operating the terminal 112, and grasps that the medical image of the patient is registered in the patient information database 113. Subsequently, the doctor on the requesting side accesses a management server 120 to issue a diagnosis request to a doctor in charge of the diagnosis who is a doctor on a requested side. The management server 120 transmits the medical image of the patient to be diagnosed and a Web page in which any other information about the diagnosis request is to be written. The terminal 112 receives this Web page and displays it. The terminal 112 then transmits the medical image and the information about the diagnosis request to the management server 120. The management server 120 registers the information about the diagnosis request including the received medical image in the order management database.

In step S912, the management server 120 reads out diagnosing doctor information contained in the diagnosis request information, and delivers the information about the diagnosis request to the diagnosing doctor. The information about the diagnosis request may be delivered as e-mail or transmitted as a Web page. In the case of a Web page, when the diagnosing doctor accesses the management server 120, the information is transmitted. Assume that the diagnosing doctor A is designated by the doctor on the requesting side.

In step S913, a terminal 131A of a diagnosing doctor A receives the diagnosis request from the management server 120. The diagnosis request is notified by, for example, e-mail. On the display of the terminal 131A, the delivered medical image and the diagnosis request information are displayed in accordance with operation by the diagnosing doctor A. For example, a link is written in the text of diagnosis request e-mail. When this link is clicked, the browser is started to display a Web page which corresponds to the link and is provided by the management server 120. This Web page contains the contents of the diagnosis request and a thumbnail of the medical image. The diagnosing doctor A clicks the displayed thumbnail image to enlarge the medical image, and properly executes a diagnosis on the basis of the information of the diagnosis request.

In step S914, the diagnostic terminal 131A acquires reference states indicating, for example, whether the medical image was referred to, the duration of reference, key operation at the time of reference, and mouse operation at the time of reference, and transmits the acquired information concerning the acquired reference states (reference state information) to the management server 120. The management server 120 stores the received reference state information in an order management database 121. Note that reference states may be acquired by the management server 120. Assume that when a thumbnail image of a medical image is clicked, the corresponding medical image is displayed on the terminal 131A. In this case, by monitoring a read of the medical image by the terminal 131A, the reference states of the medical image can be acquired by the management server 120.

When the diagnosis is completed by the diagnosing doctor A in step S915, the terminal 131A transmits the diagnosis result input by the diagnosing doctor A to the management server 120. The diagnosis result may be transmitted to the management server 120 through e-mail, a Web page, or the like.

In step S916, the management server 120 receives the diagnosis result and determines, on the basis of the stored reference states of the medical image, whether the image has been properly diagnosed. For example, this determination is made on the basis of the information indicating whether reference was made, the duration of reference, key operation at the time of reference, and mouse operation at the time of reference which are contained in the reference state information. If the medical image was not referred to or the duration of reference is shorter than a predetermined period of time, and it is highly possible that a proper diagnosis has not been done, it is determined that the image was not properly diagnosed. Assume that the predetermined period of time is the shortest time for a sufficient diagnosis and is registered in the management server 120 in advance. If it is determined that the medical image was properly diagnosed, the flow advances to step S917. Otherwise, the flow advances to step S919.

In step S917, the management server 120 accepts the diagnosis result transmitted from the terminal 131A, extracts the doctor on the requesting side from the order management database 121, and transfers the diagnosis result to the extracted doctor on the requesting side. A notification means for the diagnosis result may be e-mail or a Web page.

In step S919, the management server 120 issues a re-diagnosis request to the diagnosing doctor. The re-diagnosis request may be transmitted as a Web page or may be transmitted by e-mail. By receiving the re-diagnosis request, the diagnosing doctor A can grasp that he/she has transmitted an improper diagnosis result.

In the above embodiment, after a diagnosis result is returned from the diagnostic terminal 131A to the management server 120, the management server 120 determines the validity of the diagnosis. However, the diagnostic terminal 131A may store the reference states of the medical image, and determine the validity of the diagnosis in accordance with the stored states. For example, the terminal 131A may acquire reference states by executing a reference state acquisition program installed in the diagnostic terminal 131A in advance or may acquire reference states by executing a script transmitted from the management server 120 on a virtual machine realized by the browser.

In this case, the validity of a diagnosis can be checked before the diagnosis result is returned. This can prevent the diagnosing doctor from erroneously returning the diagnosis result without referring to the medical image, thereby reducing the load on the management server 120 which is imposed thereon when it returns useless diagnosis result.

As described above, the use of the remote diagnosis system according to this embodiment makes it possible to determine whether the diagnosing doctor has made a diagnosis by properly referring to the medical image delivered from the server, thus executing a remote diagnosis using a highly reliable medical image.

The above embodiment is based on the premise that substantially similar image display systems are installed on the requesting side and diagnosing side. However, the image display apparatus on the requested side (diagnosing side) may differ from that on the requesting side. If the performance of the image display apparatus on the requested side is lower, in particular, a diagnosis image may not be properly displayed, resulting in a trouble in the diagnosis.

This embodiment therefore provides a remote diagnosis system which can issue a diagnosis request to hospital facilities capable of properly displaying diagnosis images. This embodiment provides an accompanying advantage that even if a requesting side has no special acquaintance with a diagnosing side, the requesting side can reliably request the diagnosing side to make an image diagnosis.

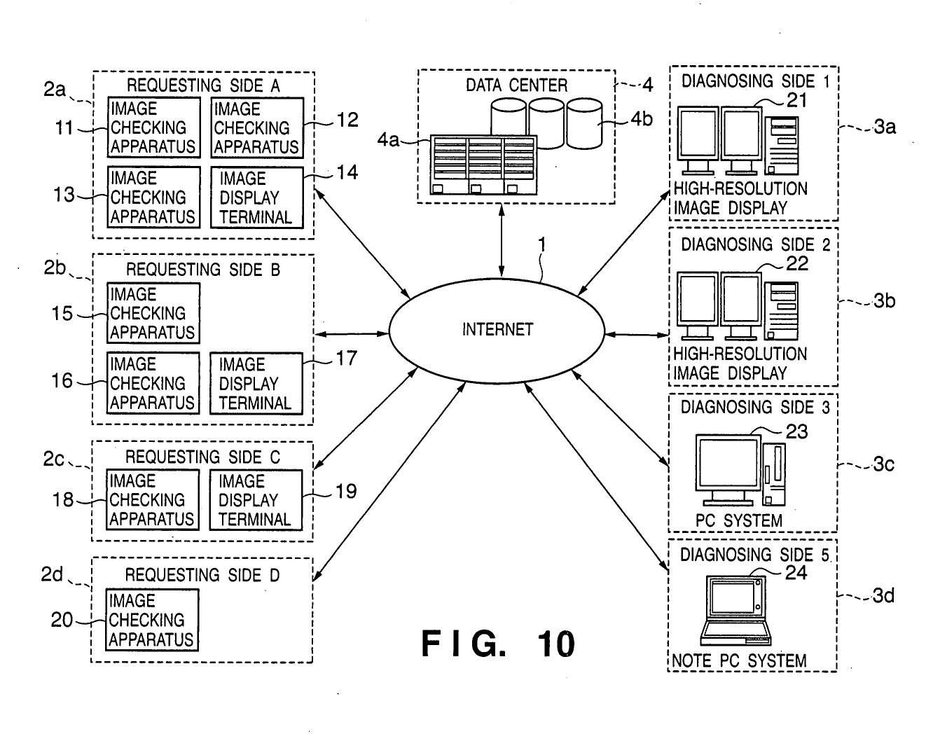

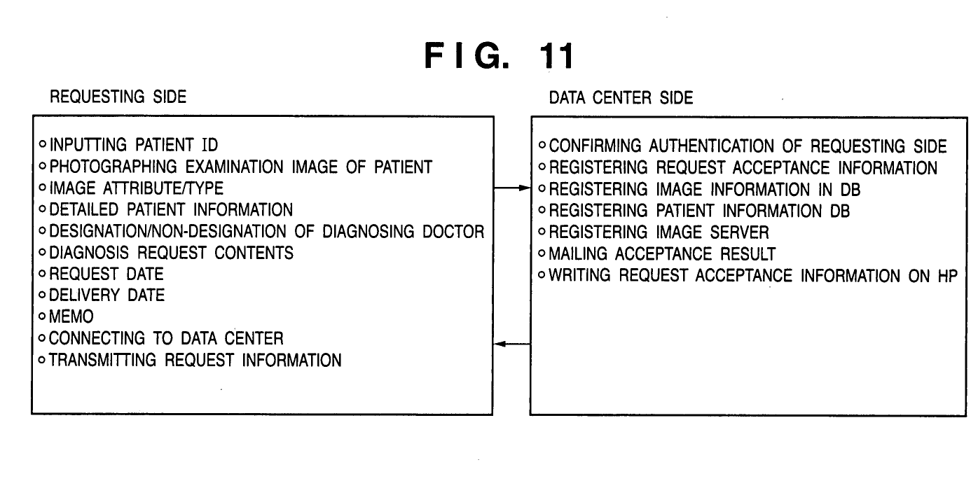

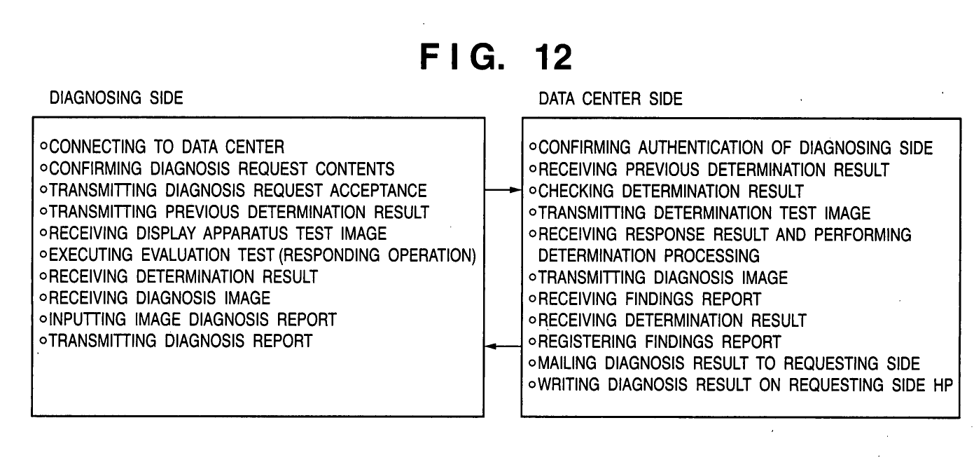

FIG. 10 is a view for explaining a remote image diagnosis system according to an embodiment of the present invention. FIG. 11 is a view showing the flow of information transmission/reception to be done between a requesting side and a data center. FIG. 12 is a view showing the flow of information transmission/reception to be done between a data center 4 and a diagnosing apparatus.

Referring to FIG. 10, reference numeral 1 denotes the Internet; 2a, a requesting side A; 2b, a requesting side B, 2c, a requesting side C, 2d, a requesting side D; 3a, a diagnosing side 1; 3b, a diagnosing side 2; 3c, a diagnosing side 3; 3d, a diagnosing side 4; and 4, the data center. They are connected to each other through the Internet 1. An image server 4a and database 4b are installed in the data center 4. The image server 4a has a function similar to that of the management server 120 described above. The database 4b has a function similar to that of the database 121 described above.

Requesting sides will be described. The requesting side A is a university hospital; the requesting side B, a health-screening center; the requesting side C, an ophthalmologic practitioner; and the requesting side D, a medical practitioner. Image-associated apparatuses are installed in the respective facilities. Image checking apparatuses 11 and 15 are, for example, an X-ray imaging apparatus and CT. An image checking apparatus 12 is an ultrasound diagnostic apparatus or the like. Image checking apparatuses 13, 16, 18, and 20 are the fundus camera 111 described above and the like. Reference numerals 14, 17; and 19 denote image display terminals. Although each image checking apparatus may have a display terminal, a description thereof will be omitted.

Diagnosing sides will be described. The diagnosing side 1 is a university hospital which has a medical specialist associated with X-ray images; the diagnosing side 2, a community medical center; the diagnosing side 3, a fundus image specialist; and the diagnosing side 4, an ophthalmologist or the like. Image display terminals are installed in the respective facilities. Note that the image display apparatuses correspond to diagnostic terminal apparatuses 131A to 131C having display apparatuses. Referring to FIG. 10, reference numerals 21 and 22 denote high-resolution image displays (monochrome; 4,000,000 to 5,000,000 pixels); 23, a high-resolution color display (2,000,000 to 3,000,000 pixels); and 24, a PC system having a 1,000,000-pixel color display.

In the university hospital as the requesting side A, the morbid portions of patients are photographed by using various image checking apparatuses every day. A large-scale hospital like a university hospital has its own image server and database installed within the facilities, and doctors in the hospital take charge of diagnosing general images.

Patients having unusual diseases are often sent to a university hospital or the like. At times, such a patient cannot be diagnosed within the hospital, or an opinion from a third party specialist is required. In such a case, a corresponding image is transmitted to the data center 4 through a communication adapter (not shown) and the Internet 1 to make a remote diagnosis request.

When a terminal apparatus in the university hospital is connected to the data center 4, authentication processing is executed. For example, the ID and password of the university hospital and the user ID and password of the operator are transmitted to the data center 4 to be authenticated. Obviously, the security of communicated data is ensured by encryption or the like.

On the image display terminal 14, the operator selects a medical image for which a remote diagnosis is to be requested, and inputs the type of image checking apparatus, the image number, image attributes (the photography date, image size, image type, and the number of gray levels), a diagnosis request purpose indicating a specific kind of diagnosis for each image, the patient ID, patient information, a request date, and a designated diagnosing doctor (more than one in some cases). The input information is transmitted as diagnosis request information.

The medical image and diagnosis request information received by the data center 4 are stored in the image server 4a and database 4b. In registering these pieces of information, the data center 4 issues a request receipt number, password, and the like, and stores the medical image and diagnosis request information in the database 4b in correspondence with each other. At the same time, the data center 4 transmits the issued request receipt number and password to the requesting side. The data center 4 also registers request contents, a progress, and the like on its Web page. When the request receipt number and password are properly input from the requesting side, the data center 4 transmits a corresponding Web page to the requester. This allows the requester to see the request contents, the progress, and the like.

In the health-screening center as the requesting side B, resident medical examinations and company medical examinations are performed by using the various image checking apparatuses 15 and 16. The medical images obtained by the checking apparatuses 15 and 16 are transmitted to the data center 4 through the Internet and a communication adapter (not shown) installed in the requesting side B, and the images and diagnosis request information are stored in the image server 4a and database 4b.

Information transmitted to the data center 4 will be described in detail. Such information includes pertinent information such as the ID and password of a health-screening center, the ID and password of an operator, an examination date as information concerning an image to be transmitted, the ID of a person to be examined, an examination place, the name of a company which takes a company examination, the type of image checking apparatus, an image number, image attributes (a photography date, image size, image type, and the number of gray levels), a request purpose indicating a specific type of diagnosis for each image, a request date, the deadline of a diagnosis result report, diagnosis destination information, storage/non-storage of the image, and an image storage period.

When the receiving operation is completed on the data center 4 side, a request receipt number, a password, and the like are issued and transmitted to the requesting side. The request contents, a corresponding progress, and the like are reflected on Web page from the data center 4 which can be browsed from the requesting side. The requester can browse the request contents, the progress, and the like by using a browser.

The ophthalmologic practitioner as the requesting side C has executed fundus photography by using the image checking apparatus 18. The photographed image is transmitted to the data center 4 and registered, together with information pertinent to the patient, in the image server 4a and database 4b. The pertinent information transmitted together with the image is similar to that described above. The doctor as the requesting side C accesses the Web page provided by the data center 4 from the image display terminal 17 through the Internet 1, and logs in to the data center 4 by inputting the user ID and password, thereby making a diagnosis while displaying the photographed image on the display of the image display terminal 17 or giving an explanation to the patient.

If the photographed image is an image associated with a disease falling outside the realm of the specialty of this doctor, he/she preferably obtains a second opinion from another specialist. The doctor therefore accesses the data center 4 from the image display terminal 17, selects a pre-registered image, and inputs diagnosis request information such as the presence/absence of a designated diagnosing doctor and a request purpose indicting a specific type of diagnosis. The doctor then registers the image diagnosis request in the data center 4. In this case, the data center 4 issues a request receipt number and the like concerning the image diagnosis request and stores them in the database 4b, and transmits the request receipt number and the like to the requesting side C.

The medical practitioner as the requesting side D practices internal treatment, and performs fundus photography for a diabetic patient by using the image checking apparatus 20 to diagnose the state of his/her retinopathy. In this description, it is assumed that the physician requests other doctors to perform image interpretation for all fundus images. Therefore, request information such as the patient ID and a request purpose indicating a specific type of diagnosis is added to each image photographed by the image checking apparatus 20, and the resultant information is transmitted to the data center 4. The image and additional information transmitted to the data center 4 are registered in the above manner.

The processing performed on the data center 4 side includes the processing of receiving an image and request information from a requesting side, the processing of registering the received image in the image server 4a, the processing of registering the accompanying request information in the database 4b, and user authentication processing at the time of connection between the requesting side the data center 4. A detailed description of this processing will be omitted.

The image server 4a manages images on a user basis, and manages the attribute information of each image in the database 4b. The word “user” means a hospital, a doctor, a patient, or the like. The image server 4a also takes charge of image registration processing, read processing, and conversion processing, e.g., extracting a registered image in accordance with a request from the database 4b, converting the format of a registered image into a format complying with a system on the diagnosing side, and reducing the size of a registered image to an image size corresponding to the contents of a diagnosis.

The database 4b is a database system including a plurality of databases. The database 4b includes, for example, a database in which key codes for identifying images registered in the image server 4a, pieces of identification information for identifying requesting sides and users on the requesting sides, and pieces of information for identifying diagnosing sides and diagnosing users are registered, a database in which schedules on the diagnosing side are registered, a database in which image attribute information is registered, a database which manages request receipt dates, the deadlines of diagnoses, and delay information of diagnoses, and a database which charges for a diagnosis request and storage of an image. These databases may be similar to the above database 121. The main function of the data center 4 is associated with the management server 120 described above.

The database 4b periodically searches for an image diagnosis request to extract the corresponding requested case, and generates a list of doctors or hospitals serving as diagnosing sides in consideration of designation/non-designation of diagnosing sides, the schedules of diagnosing sides, request contents, a diagnosis purpose, and the like. The database 4b then registers the generated list on a Web page which can be browsed from the diagnosing sides. The image server 4a then transmits e-mail as a diagnosis request to the diagnosing sides in accordance with the priority levels designated in the diagnosing side list.

A terminal apparatus (e.g., the PC system 12) on the diagnosing side accesses the Web page having the diagnosis request contents on the basis of the URL written in the request mail from the data center 4, thereby downloading the Web page associated with the diagnosis request contents and displaying them on the display. The doctor on the diagnosing side refers to the displayed request contents and determines whether to accept the diagnosis request. Obviously, user authentication or the like is performed when a terminal apparatus on the diagnosing side accesses the data center 4.

Upon reception of an acceptance request from a terminal apparatus on the diagnosing side, the data center 4 determines, in accordance with image information and diagnosis purpose to be transmitted to the diagnosing side, whether a display apparatus on the diagnosing side is appropriate. More specifically, the data center 4 searches the database 4b in which the determination result on the display apparatus on the diagnosing side is registered in advance, and stores the corresponding determination result on the diagnosing side in a temporary storage area. The data center 4 then determines whether this determination result coincides with the purpose of the diagnosis request to be made. If they coincide with each other, the data center 4 connects to the display apparatus on the diagnosing side to read out the previous determination result information stored in the cache of the display apparatus. The database 4b determines whether this information coincides with the information stored in the temporary storage area.

Upon determining that the diagnosis request contents such as the diagnosis purpose differ from the previous contents, the data center 4 executes a display performance test. In addition, if the interval between the previous determination and the current determination exceeds a predetermined period, a display performance test may be executed. The predetermined period can be adjusted from the data center 4. For example, on a diagnosing side where the display apparatus is periodically maintained, the predetermined period may be set to three months, whereas for a user who cannot maintain or inspect the display apparatus by himself/herself, e.g., a private diagnosing doctor, the predetermined period may be set to one month or the like.

If the interval between the previous determination and the current determination exceeds the predetermined period, a test image for executing a display performance test on the display apparatus on the diagnosing side is transmitted to the diagnosing side, and it is determined whether an image diagnosis can be properly done.

If no determination result is recorded on the cache of the diagnosing side apparatus, a display performance test image is transmitted as in the above case. If all conditions such as a diagnosis purpose and image attributes are not satisfied even though a diagnosis result is recorded, a display performance test image for executing a display performance test is transmitted.

If the display apparatus oh the diagnosing side passes the display performance test, the test result is registered in the database 4b. In addition, the test result is encrypted and stored in the cache of the display apparatus.

The system on the diagnosing side then downloads a diagnosis image from the image server 4a. The downloaded image is set to the original size or reduced in accordance with the diagnosis purpose. For an examination image and the like, a precise diagnosis is not required, and it is only required to determine whether there is a suspicion of disease. In a medical examination, since a large quantity of examination images must be diagnosed at once, the examination images are lossy-compressed and downloaded into the system on the diagnosing side. Note that when a diagnosing doctor terminal issues a request to transmit an original examination image or an image with a size one step larger than the original size, the image server 4a may transmit the requested examination image to the terminal on the diagnosing side.

Since a diagnosis report in which findings are input is also transmitted from the image server 4a, together with the image, the system on the diagnosing side inputs a finding result in parallel with a diagnosis. Every time the diagnosing side performs image processing or changes the range of gray levels during inputting of findings to make the image easy to read, information associated with the image processing is stored in the finding entry area of a diagnosis report. These pieces of information may be converted into data invisible to the diagnosing doctor or made unchangeable.

When all findings are input, the system on the diagnosing side transmits the diagnosis report to the data center 4. At the time of transmission, the system may also transmit the determination result of the display test. Comparing information concerning the date and time when the findings were input with the date and time when the image was downloaded makes it possible to determine whether the interval between the instant at which the image was downloaded and the instant at which the findings were transmitted exceeds a given period of time.

In addition, the display test result on the display apparatus used for the diagnosis is attached to the diagnosis report. Comparing this attached display result with the display test result under the environment at the time of downloading allows the data center 4 to determine whether the diagnosis image has been moved from the system used for downloading to another system.

If a diagnosis report is transmitted during an image diagnosis, the data center 4 counts the number of images uninterpreted. This number is displayed on a predetermined Web page on the diagnosing side. In such a case, the remaining part of the diagnosis can be executed on the diagnosing side later, and a diagnosis report can be transmitted again.

Assume that when a diagnosing doctor diagnoses a medical image, the result is an unclear case, or he/she lacks confidence in the diagnosis. In this case, it is important to hear an opinion from another specialist. In such a case, the diagnosing doctor can designate a new diagnosing doctor by the same procedure as that on the requesting side described above. If a plurality of diagnosing doctors are designated in advance, the diagnosing doctor may issue a diagnosis request to another diagnosing doctor again. In this case, since the medical image itself has already been registered in the data center 4, the new diagnosing doctor can display the medical image to be diagnosed and diagnose it by only selecting the registered image through the terminal apparatus of the diagnosing doctor. In this case as well, return processing of the above diagnosis result, reference state acquisition processing, and the like are executed.

When the data center 4 issues an image diagnosis request to the diagnosing side and then receives a diagnosis report from a terminal apparatus on the diagnosing side, the diagnosis result is copied on a finding table on the corresponding requesting side. The data center 4 transmits e-mail indicating the completion of the remote diagnosis to the doctor on the requesting side or hospital.

When a terminal apparatus on a requesting side accesses a predetermined Web page, the data center 4 displays the contents of a predetermined table including a finding result, the number of images unprocessed, and the like as a Web page. For example, at the next examination of a patient, the requesting side D can make an appropriate treatment or refer the patient to another necessary doctor upon giving an explanation to the patient on the basis of the diagnosis report and key image.

FIG. 11 is a view showing a transmission/reception sequence between a requesting side and the data center. Although FIG. 11 shows time-series procedures from top down in the blocks, processing need not always be performed according to the procedures.

On the requesting side, when a patient is to be photographed, a patient ID is input first to a checking apparatus such as the fundus camera 111. In a medical examination or the like, an examination number is also input. The checking apparatus then acquires image data. At this time, the checking apparatus adds information such as image attributes and type and the patient ID (or the examination number) to the image. These pieces of information may be directly added inside the image or attached as a pertinent file to the image data.

Detailed patient information is then added from the patient information database 113 on the diagnosing side, as needed. Thereafter, for example, the requesting side designates a diagnosing doctor, or makes a designation to leave the choice to the diagnosing side registered in the data center 4. A diagnosis purpose is selected next. For example, the requesting side selects one of the following diagnosis purposes: a diagnosis for screening, a diagnosis concerning a specified disease such as diabetic retinopathy, and a specialized, precise diagnosis concerning an X-ray image or MR image, thus inputting diagnosis request contents. A request date is automatically input from timepiece data in the data center 4. The deadline of a diagnosis result is also designated. In addition, memo information is input if any. When the request start button 402 is clicked, the input or selected information and image information are transmitted to the data center 4 through the Internet 1.

Assume that the image is an examination image. In this case, if only an examination number is input at the time of photography and photography is performed, information registered in advance in the patient information database 113 is read out and added by the checking apparatus. The checking apparatus transmits images and the like in bulk to the data center 4 at predetermined time intervals. When the terminal apparatus on the requesting side is connected to the data center 4, the data center 4 receives the ID, password, or the like of the hospital on the requesting side or doctor from the terminal apparatus, and collates it with the information about the hospital on the requesting side or doctor which is registered in advance in the database.

The received information accompanying the diagnosis request is registered in the database 4b. The image information is registered in the image server 4a. Upon completion of the registration, e-mail concerning the acceptance result is transmitted to the requesting side. In addition, the acceptance information is written on a Web page which can be accessed from the requesting side. The data center 4 retrieves a diagnosing hospital or doctor on the basis of the diagnosis request contents, and transmits e-mail of a diagnosis request to the corresponding diagnosing side. In addition, the request information is written on a Web page which can be accessed from the diagnosing doctor.

FIG. 12 is a view showing a transmission/reception sequence between the data center and an apparatus on a diagnosing side. FIG. 12 shows time-series procedures from top down in the blocks. A terminal apparatus on the diagnosing side accesses a Web page having request information on the basis of request mail from the data center 4. At this time, the data center 4 executes authentication processing on the diagnosing side.

The data center 4 connects to a display apparatus on the diagnosing side and receives the previous determination result. The data center 4 then compares this determination result with the contents registered in the database 4b.

For example, the data center 4 checks the time difference between the previous determination and the current determination, and determines whether the previous diagnosis purpose coincides with the current diagnosis purpose, and the attributes of the previous image coincide with those of the current image. If the display apparatus on the diagnosing side satisfies conditions such as the current diagnosis purpose, the transmission of a test image to the display apparatus is skipped, and downloading of a diagnosis image is executed. If the conditions do not coincide with each other, since a test is required again, an image for a performance test on the display apparatus is transmitted to the diagnosing side.

The data center 4 waits for a response from the diagnosing side and determines on the basis of the result whether the display apparatus matches the current diagnosis purpose. If no problem arises in this performance test result, the diagnosis image is downloaded. If the display apparatus does not match the diagnosis request purpose, the data center 4 notifies the apparatus on the diagnosing side of the corresponding information. Note that the display performance of the display apparatus on the diagnosing side has deteriorated, it is determined that the apparatus is faulty, and the diagnosing side is notified of the failure contents.

In addition to the diagnosis image, a diagnosis repot on which no findings are written is transmitted from the data center 4 to the diagnosing side. Upon reception of the diagnosis report on which findings on the diagnosing side are written, the data center 4 registers the contents in the database 4b, and notifies the requesting side of the completion of the diagnosis by mail. At the same time, the result contents are written on a home page which can be accessed by the requesting side.

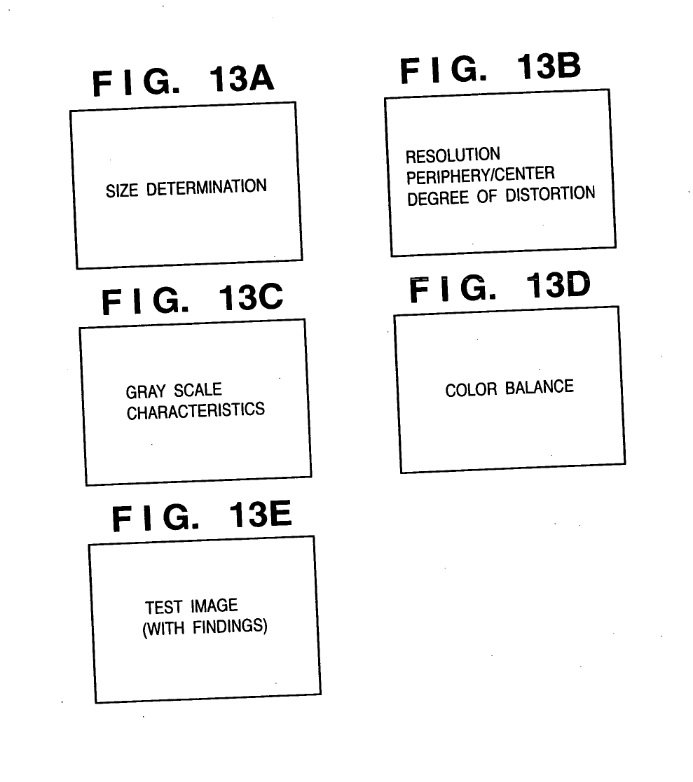

FIGS. 13A to 13E respectively show display performance test images which are downloaded into the display apparatus on the diagnosing side and displayed.

FIG. 13A shows an image for determining an image size. For example, a plurality of size determination images may be prepared on a photographing apparatus basis or photographing method basis or in accordance with a reference purpose or a request purpose on the requesting side. The data center 4 selects one of the plurality of images in accordance with the application purpose.

For example, an X-ray image is constituted by 4,000,000 to 5,000,000 pixels. When, therefore, the purpose is to perform a precise diagnosis, a display apparatus on a diagnosing side must display the X-ray image without reduction in size. In contrast, when the diagnosis purpose is to perform a screening diagnosis, it suffices if image interpretation can be so performed as to determine whether there is a suspicion of a disease to be diagnosed. Therefore, no problem arises even if the image is displayed in a reduced size of about ½ to ¼. Fundus photography images include, for example, an image constituted by 2,000,000 to 3,000,000 pixels, which is obtained by photographing up to minute blood vessels by fluorescence fundus photography. The required display size of this image also varies depending on the diagnosis purpose. For example, a color image photographed in a periodic medical examination generally has about 300,000 to 400,000 pixels, and hence may be displayed in a display size similar to SVGA (800×600).

An image for testing the display size of a display apparatus is displayed with the origin (0, 0) at the upper left on the display screen. Therefore, whether the display apparatus used on the diagnosing side is suited to the test image can be determined by making the operator input information indicating whether symbols located at the lower side or right end can be displayed or making the operator press a switch placed on the lower side or right side. Alternatively, this determination may be done by causing the data center 4 to access the display apparatus of the terminal on the diagnosing side and read out information concerning the displayable size stored in a cache memory or the like of the display apparatus.

FIG. 13B shows a test image for determining the resolution or the degree of distortion of a display apparatus. For a display apparatus using a CRT, it is determined whether the focus becomes less sharp or the resolution decreases or distortion increases at the periphery due to a deterioration in deflection characteristics. A test image includes necessary resolution charts at a plurality of positions. The operator is prompted to input, from the keyboard, information indicating whether the charts can be read. The resolution charts are arranged at the center and four corners of the display apparatus. These charts may be designed to simultaneously allow determination of distortion. As such test images, test images corresponding to display sizes are prepared to be selectively used depending on the diagnosis purpose.

FIG. 13C shows a test image for determining up to which number of gray levels the display apparatus can display an image. Such test images may be respectively prepared for color and monochrome displays. For a color display, since RGB 8-bit data is a standard, only one type of gray scale image is prepared. However, a plurality of patterns are prepared. For a monochrome display, test images with 8, 10, and 12 gray levels are prepared. One of these images is selected depending on the diagnosis purpose.

FIG. 13D shows a test image for checking the color balance of a display apparatus. This image is used to determine whether the color temperature setting or color balance of the display apparatus is one-sided, or the balance has deteriorated due to due to a deterioration in quality or a change over time. This image allows the operator to confirm whether a predetermined color is properly displayed with a predetermined combination of R, B, and G. On the basis of the response to the confirmation, this determination is made. This test image is selected when a color image is selected as a diagnosis image.

FIG. 13E shows a test image with findings based the execution of one diagnosis in the past. This test image is downloaded and displayed when the test steps shown in FIGS. 13A to 13D are cleared. The operator is then prompted to select one of three to five choices which indicates the specific diagnosis content of the test image. If the selected diagnosis content matches the test image, it is determined that the selection is correct. This operation is executed for a plurality of test images. If all selections are correct, it is determined that the display apparatus on the diagnosing side matches the diagnosis request purpose.

When this determination is executed, the result is written in a cache area in the system on the diagnosing side from the data center 4. For example, a file is generated, which includes the date and time of pass determination, the user ID to log in to the data center 4, and pass contents. This file is written after being encrypted to inhibit a user on the diagnosing side from reading.

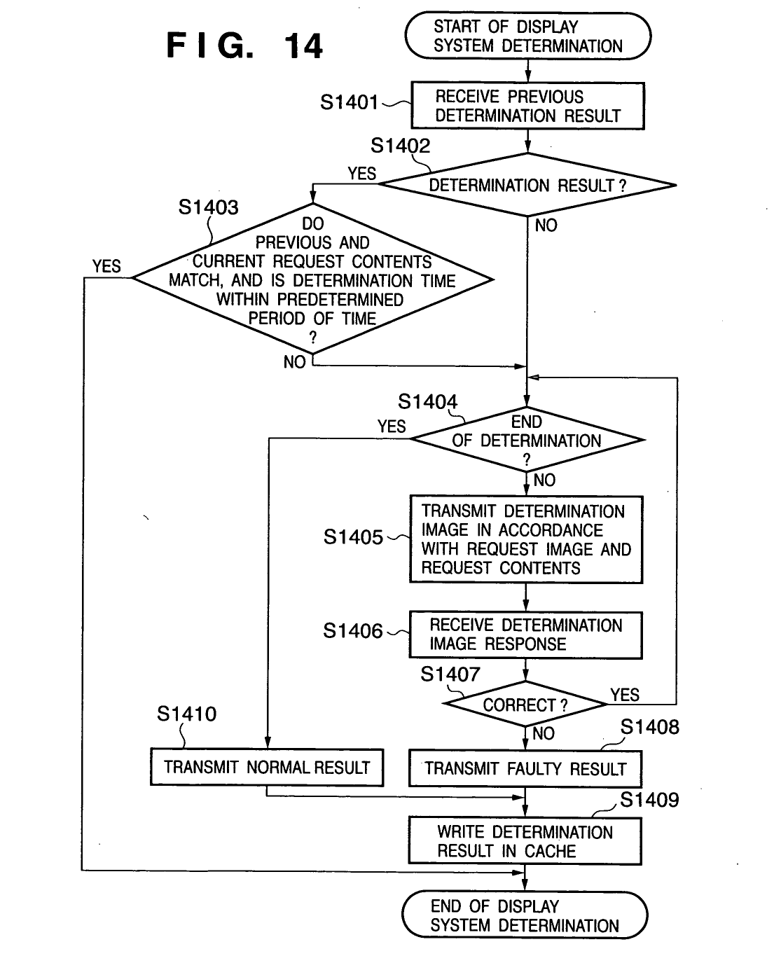

Determination processing in display processing will be described in detail next. FIG. 14 is a flow chart associated with determination processing for a display apparatus.

In step S1401, the data center 4 connects to a display apparatus on a diagnosing side and receives the previous determination result.

In step S1402, the data center 4 checks the received determination result. If the previous determination result exists, the flow advances to the step S1403. Otherwise, the flow advances to step S1404.

In step S1403, the data center 4 determines, for example, whether the previous determination time is within a predetermined period, and the previous diagnosis purpose coincides with the current diagnosis purpose. If these conditions are satisfied, the display apparatus determination flow is terminated. Otherwise, the flow advances to step S1404 to perform a performance test on the display apparatus again.

In step S1404, the data center 4 determines whether the determination processing is completed. If YES in step S1404, the flow advances to step S1410. If NO in step S1404, the flow advances to step S1405.

In step S1405, the data center 4 transmits a determination image to the diagnosing side. The determination image is registered in, for example, the database 4b in advance. In this step, the data center 4 properly selects a determination image in accordance with the type of request image and diagnosis purpose. For example, the database 4b stores in advance the correspondence between determination images and diagnosis purposes. The data center 4 reads out a determination image in accordance with the diagnosis purpose received from a requesting side.

In step S1406, the data center 4 receives a response from the diagnosing side with respect to the transmitted determination image.

In step S1407, the data center 4 determines whether the response to the determination image is correct. If the response is correct, the flow returns to step S1404 to perform the next determination step. If the response from the diagnosing side is incorrect, the flow advances to step S1408.

In step S1408, the data center 4 transmits information indicating a faulty state of the display apparatus on the diagnosing side to the diagnosing side. The contents of this information depend on the determination result in the determination step. For example, the diagnosing side is notified of a message like “The screen size is insufficient to display the diagnosis image.”, “The resolution of the screen is insufficient to diagnose this image.”, or “There is a problem in color balance. Please adjust it.” The processing in step S1408 is performed when the request image does not match the display apparatus on the diagnosing side, or it is determined that the display apparatus on the diagnosing side is faulty. In step S1409, the data center 4 transmits the determination result to the diagnosing side. This determination result is written in the display apparatus on the diagnosing side.

If all the determinations are normally completed, the data center 4 notifies the diagnosing side of the determination result in step S1410. For example, a message like “The performance test on the display apparatus is finished. The performance requirement for diagnosis is satisfied.” is transmitted to the diagnosing side. In step S1409, the data center 4 transmits a result indicating whether the display apparatus has passed the test, and writes it in the display apparatus on the diagnosing side. The contents to be written include the date, request image information, request contents, and determination result, and are stored in the form of encrypted information. The diagnosing side cannot therefore see, correct, or change the contents.

In the above embodiment, a display apparatus on a diagnosing side is evaluated/determined on the basis of an examination image and a diagnosis purpose on the requesting side. If, however, the requesting side is to transmit only image information, the requesting side can determine diagnosis request contents on the basis of the file size, image size, color information, and the like which can be known from the image file, select a determination image that satisfies the contents, and transmit it to the diagnosing side.

If, for example, a given image is a monochrome image with 4,000,000 pixels or more, it is determined that a precise diagnosis of an X-ray image is requested, and an evaluation/determination image that can guarantee the request is selected. If a given image is a monochrome image with 4,000,000 pixels or less and 1,000,000 pixels or more, it is determined that a precise diagnosis of a fluorescence fundus image is requested, and an evaluation/determination image that can guarantee the request is selected. If a given image is a color image with 500,000 pixels or less, it is determined that a diagnosis of a screening fundus image is requested, and an evaluation/determination image that can guarantee the request is selected.

In the above embodiment, the data center 4 transmits a determination image to determine the performance of an image diagnosis display apparatus on a diagnosing side. However, the diagnosing side may be made to download a program for diagnosing/determining a display apparatus and execute the determination program when it connects to the data center 4. For a display apparatus used for an image diagnosis, since the contents need to be updated once or at predetermined intervals, the time required to download a determination image can be saved.

In the above embodiment, examination images on a requesting side are managed once by the data center 4 connected to the Internet, and the data center 4 issues requests to diagnosing sides. However, the present invention may be applied such that the data center 4 is installed within large-scale facilities such as a health screening-center or large hospital, and a remote diagnosis of an examination image generated within the facilities is requested outside the facilities.

As has been described above, according to this embodiment, a remote diagnosis of a medical image of a patient can be efficiently performed. In addition, since a diagnosis request is issued under the condition that a display apparatus used for a diagnosis on a diagnosing side has passed a performance test, the diagnosis result is guaranteed to some extent. This allows the requesting side to trust the diagnosis result more than in the prior art.

In addition, a request image can be transmitted to a diagnosing side after being reduced in size in accordance with a diagnosis purpose, thereby improving the response of an image diagnosis.

Furthermore, in accordance with a diagnosis purpose, an image with findings is presented to a diagnosing doctor to make he/she determine whether the diagnosis result match the findings. This makes it possible to evaluate the performance of the display apparatus used for the diagnosis and also can check the image interpretation ability of the diagnosing doctor, thus contributing to the maintenance of a relationship of trust between the requesting side and the diagnosing side.

The present invention can be applied to a system constituted by a plurality of devices, or to an apparatus comprising a single device. Furthermore, it goes without saying that the invention is applicable also to a case where the object of the invention is attained by supplying a program to a system or apparatus.

As many apparently widely different embodiments of the present invention can be made without departing from the spirit and scope thereof, it is to be understood that the invention is not limited to the specific embodiments thereof except as defined in the appended claims.

1. A remote diagnosis system which helps a doctor on a requesting side request a remote diagnosis to a doctor on a requested side to execute the remote diagnosis, comprising:
component for storing examination data of a patient transmitted from a terminal on a requesting side;
component for selecting a doctor on a requested side on the basis of the examination data of the patient and preset priority levels;
component for transmitting a diagnosis request to the selected doctor on the requested side;
component for transmitting the registered examination data when being accessed by the selected doctor on the requested side from a terminal on the requested side;
component for storing diagnosis result data transmitted from the terminal on the requested side; and
component for transmitting the diagnosis result data to the terminal on the requesting side.

2. The remote diagnosis system according to claim 1, further comprising component for providing a terminal of the doctor on the requested side with a menu window for selecting at least two of registration processing of diagnosis result data, diagnosis entrustment processing for another doctor, and re-examination request processing for the requesting side.

3. The remote diagnosis system according to claim 1, further comprising component for providing the terminal on the requesting side with a menu window for selecting whether to give the doctor on the requested side a right to change the priority levels.

4. The remote diagnosis system according to claim 1, further comprising:
component for providing the terminal on the requesting side with a selection menu for selecting whether to permit the doctor on the requested side to entrust another doctor with a diagnosis, and
component for providing the terminal on the requested side with a menu window for selecting entrustment of a diagnosis to another doctor, only when the doctor on the requested side is permitted to perform entrustment of a diagnosis.

5. The remote diagnosis system according to claim 1, further comprising:
component for counting the number of cases in which a cooperative diagnosis is requested to the doctor on the requested side,
component for counting the number of cases in which the doctor on the requested side entrusts another doctor with a diagnosis, and
component for changing the priority levels on the basis of at least one of the counted numbers.

6. A remote diagnosis method which makes a doctor on a requesting side request a remote diagnosis to a doctor on a requested side to execute the remote diagnosis, wherein:
examination data of a patient transmitted from a terminal on a requesting side is stored,
a doctor on a requested side is selected on the basis of the examination data of the patient and preset priority levels,
a diagnosis request is transmitted to the selected doctor on the requested side,
the registered examination data is transmitted when access is made by the selected doctor on the requested side from a terminal on the requested side,
diagnosis result data transmitted from the terminal on the requested side is stored, and
the diagnosis result data is transmitted to the terminal on the requesting side.

7. A remote system which performs a remote diagnosis through a doctor on a requesting side and a doctor on a requested side, comprising:
component for storing diagnosis/treatment information including a medical image transmitted from a terminal on a requesting side;
component for delivering the medical image to a terminal on a requested side;
component for acquiring information concerning a reference state of the terminal on the requested side with respect to the delivered medical image; and
component for storing the acquired information concerning the reference state.

8. The remote diagnosis system according to claim 1, further comprising component for transmitting a re-diagnosis request to the terminal on the requested side in accordance with the information concerning the reference state.

9. The remote diagnosis system according to claim 1, wherein the information concerning the reference state includes at least one of information indicting whether the doctor on the requested side referred to the medical image, a reference time, a keyboard operation history, and a mouse operation history.

10. A method of performing a remote diagnosis through a doctor on a requesting side and a doctor on a requested side, comprising the steps of:
storing diagnosis/treatment information including a medical image transmitted from a terminal on a requesting side;
transferring the medical image to a terminal on a requested side;
receiving information concerning a reference state of the terminal on the requested side with respect to the transferred medical; and
storing the received information concerning the reference state.

11. A remote diagnosis system which makes a doctor on a requesting side request a remote diagnosis to a doctor on a requested side to execute the remote diagnosis, comprising:
component for storing a diagnosis request content transmitted from a terminal on a requesting side; and
component for evaluating a display apparatus used by the doctor on the requested side on the basis of the diagnosis request content.

12. A remote diagnosis system which makes a doctor on a requesting side request a remote diagnosis to a doctor on a requested side to execute the remote diagnosis, comprising:
component for storing a transmitted examination image of a patient and a transmitted request content;
component for setting an evaluation content for evaluating performance of a display apparatus used by the doctor on the requested side,
component for evaluating the performance of the display apparatus on the basis of the set evaluation content; and
component for transmitting diagnosis request information of the examination image to the doctor on the requested side associated with the display apparatus which has passed the performance evaluation.

13. The remote diagnosis system according to claim 12, wherein:
said component for setting the evaluation content includes
component for storing a plurality of evaluation images to be used for performance evaluation of the display apparatus,
component for selecting at least one of the plurality of evaluation images on the basis of at least one of the examination image and the request content, and
component for presenting the selected evaluation image to the display apparatus, and
said component for evaluating the performance evaluates the display apparatus in accordance with a response from the requested side.

14. The remote diagnosis system according to claim 12, wherein said component for presenting the evaluation image includes a program for presenting the plurality of evaluation images to the display apparatus.

15. The remote diagnosis system according to claim 12, wherein:
the system further comprises component for storing information concerning a previous performance evaluation result, and
the performance of the display apparatus is evaluated on the basis of the information concerning the stored previous performance evaluation result and a current diagnosis request and/or an examination image.

16. The remote diagnosis system according to claim 12, wherein:
the system further comprises
component for storing information concerning a previous diagnosis request date, and
component for determining whether a difference between the previous diagnosis request date and a current diagnosis request date exceeds a predetermined period, and
said component for evaluating the performance re-evaluates the display apparatus when the difference exceeds the predetermined period.

17. The remote diagnosis system according to claim 12, wherein:
the system further comprises component for determining whether a previous request content substantially differs from a current request content, and
the display apparatus is re-evaluated when the previous request content substantially differs from the current request content.

18. The remote diagnosis system according to claim 12, wherein:
the system further comprises
component for determining whether a previous request content substantially differs from a current request content, and
component for determining whether a difference between a previous diagnosis request date and a current diagnosis request date exceeds a predetermined period, and
said component for evaluating the performance re-evaluates the display apparatus when one of a condition that the previous request content substantially differs from the current request content and a condition that the difference exceeds the predetermined period is satisfied.

19. A remote diagnosis method which helps a doctor on a requesting side request a remote diagnosis to a doctor on a requested side to execute the remote diagnosis, comprising the steps of:
storing a transmitted examination image of a patient and a transmitted request content;
setting an evaluation content for evaluating performance of a display apparatus used by the doctor on the requested side;
evaluating the performance of the display apparatus on the basis of the set evaluation content; and
transmitting diagnosis request information of the examination image to the doctor on the requested side associated with the display apparatus which has passed the performance evaluation.
